(19) United States
(12) Patent Application Publication
(10) Pub. No.: US 2025/0312199 A1
(43) Pub. Date: Oct. 9, 2025

(54) MALE INCONTINENCE ARTICLE AND RELATED METHODS

(71) Applicants: Christopher Alan Nippert, Duluth, GA (US); Jorge Antonio Nieri, Knoxville, TN (US)

(72) Inventors: Christopher Alan Nippert, Duluth, GA (US); Jorge Antonio Nieri, Knoxville, TN (US)

(21) Appl. No.: 18/628,018

(22) Filed: Apr. 5, 2024

(51) Int. Cl. A61F 13/471; A61F 13/511; A61F 13/53; A61F 13/58; A61F 13/62

(52) U.S. Cl. CPC A61F 13/471; A61F 13/51113; A61F 13/53; A61F 13/581; A61F 13/622; A61F 2013/51117; A61F 2013/530021; A61F 2013/530518

(57) ABSTRACT

An incontinence article for a male that has a penis and a scrotum extends along a longitudinal axis. The article has a pouch and a sling that is connected to that pouch. The pouch includes one or more layers of nonwoven material and is configured to envelop the penis, with the pouch having a first open end and a second closed end that is longitudinally opposed the first open end. The sling is connected to the pouch adjacent the first open end of the pouch and includes a loop portion that is configured to fit around an underside of the scrotum and the penis of the male, with that loop portion being extensible in at least a generally circumferential direction of the loop portion.

### TECHNICAL FIELD

The present disclosure is generally related to incontinence articles and, more particularly, to incontinence articles to be worn by men.

### BACKGROUND

Conventional incontinence hygiene articles for men do not meet the necessary level of urine (and other fluids) leakage protection, while maximizing comfort during wear, discreetly, and securely. Incontinence articles for men include pads (also referred to as guards or shields), and protective underwear (also referred to as adult diapers), but they are mere adaptations of hygiene products made for women. Articles of that type designed for women, as well as those designed for men, typically include an acquisition/distribution layer (“ADL”) that absorbs and distributes urine and/or other bodily fluids discharged by the wearer and an absorbent core under the ADL that stores the urine. The anatomy of female genitalia necessitates that the ADL of incontinence articles be situated close to the wearer's body in a stationary position. In that regard, the solution to controlling female leakage has been to design articles in the form of pads that attach securely to the woman's underwear, or protective underwear that replaces the conventional cloth underwear.

By contrast, the anatomy of male genitalia, particularly the penis, is external to the man's body and moves freely. As a result of the external location of the penis, articles that fit close to the body in a stationary position are both uncomfortable and ineffective. The ADL of conventional articles adapted for men is pressed against the penis, does not move with the penis, and tends to shift during movement. Consequently, the ADL and the absorbent core disposed under the ADL are often out of place when fluid discharge occurs, thereby resulting in leakage. To reduce shifting, articles such as pads are often worn with tight-fitting briefs, but those tend to be uncomfortable for the male wearer for the reasons discussed above. And it should be noted that a large percentage of men prefer to wear loose-fitting underwear, such as boxer shorts or boxer briefs because they are deemed more comfortable. Men, accordingly, must forego the relative comfort of looser-fitting types of underwear when wearing pads.

To compensate for movement of the penis and of the article relative to the penis, pads and protective underwear for men are designed to be large, with plastic polymer and/or other film material outer covers that extend over a wide area as a way to avoid or at least minimize leakage. This fact makes those articles difficult to wear and carry discreetly. Additionally, they tend to be embarrassingly noisy when they rub against the wearer's skin and/or clothing; they are cumbersome to change, hard to manage when using the restroom, and are not environmentally friendly. Moreover, protective underwear articles are bulky to wear and are too large to carry in the man's pocket. Specifically, conventional pads may be about 12 inches long and five inches wide or larger, taking up significant room in the underwear and being too large to carry in the man's pockets.

Another problem of conventional pads and protective underwear designed for men is that the constant rubbing of those articles against the skin around the wearer's legs and lower buttocks area tends to cause skin irritation and even rashes. Yet another problem is in the changing of those articles when needed, which is also difficult. Conventional pads are affixed to underwear with adhesive and must be peeled off to change. With protective underwear, pants must be pulled down to change, or completely taken off in the case of pull-up briefs. And the difficulty is greatly enhanced for those that are confined to bed. Specifically, in such cases, a caregiver must lift up the body of the man to remove and change the protective underwear.

Articles such as those described above also make it necessary for the man's pants to be pulled down to use the restroom, which makes using stand-up urinals in public restrooms awkward, inconvenient, and embarrassing for the wearer. Additionally, articles of that type use significant amounts of plastics and non-biodegradable materials that contribute to land and ocean pollution. Yet another concern with such products lies in the fact that men do not want to be thought of as wearing women's pads or baby diapers. As a result, a large percentage of men who need leakage protection go without any, leading to an overall poor lifestyle for those men, who resultingly often isolate themselves from the rest of society.

Other conventional hygiene articles include a drip collector that suffers design flaws that make it uncomfortable to wear and largely ineffective. That type of article attaches directly to the shaft of the penis, with the attachment needing to be firm to avoid slipping off. Tight attachment causes pinching and may even disrupt blood circulation. Further, drip collectors must be taken off to use the restroom and reattached afterward. This makes using a public urinal awkward and increases the risk of pinching the penis when retightened or slipping off if not retightened to a sufficient extent. And the likelihood of the drip collector slipping off is exacerbated when the weight of the urine collected by the article reaches a predetermined level, which forces manufacturers of such articles to limit the volume of urine they are designed to hold.

Conventional drip collectors must also fit closely to the penis head, limiting the area of the absorbent core component. That is the case because should the drip collector extend too far away from the penis head, the excess material of the collector would rub against the underwear, pulling the drip collector off. For the foregoing reasons, drip collectors have weight, size, and containment level limitations, thereby offering men ineffective levels of leakage or skin wetness protection.

It would be desirable, therefore, to address these and other shortcomings described above in connection with conventional male incontinence articles.

### SUMMARY

In one embodiment, an incontinence article is disclosed for a male that has a penis and a scrotum, with the incontinence article extending along a longitudinal axis. The article has a pouch and a sling that is connected to that pouch. The pouch includes one or more layers of nonwoven material and is configured to envelop the penis, with the pouch having a first open end and a second closed end that is longitudinally opposed the first open end. The sling is connected to the pouch adjacent the first open end of the pouch and includes a loop portion that is configured to fit around an underside of the scrotum and the penis of the male, resting against the perineum, with that loop portion being extensible in at least a generally circumferential direction of the loop portion.

The pouch may include an absorbent core that, in specific embodiments, has super absorbent polymer material (“SAP”). Additionally, or alternatively, the absorbing core may include fluff pulp in an amount not exceeding about 20% by weight of that absorbent core. The loop portion may include at least one elastic or non-elastic strand that extends in the generally circumferential direction of that loop portion. The sling may include a proximal portion connected to the pouch, a pair of leg portions extending from the proximal portion, and a distal portion that spans between respective terminal ends of the leg portions in a direction transverse to the longitudinal axis of the article. The proximal, leg, and distal portions may jointly define the loop portion of the sling. The proximal portion may or may not be integrally formed with the pouch. Additionally, the leg portions and the distal portion may have respective length dimensions, with each of the leg and distal portions being extensible along those respective length dimensions.

In a specific embodiment, each of the leg portions and the distal portion includes a plurality of elastic strands extending generally along the respective length dimensions of those portions. Alternatively, the strands may be non-elastic. The leg portions and the distal portion may be integrally formed with one another. Additionally, or alternatively, the leg portions and the distal portion may be formed from a multidirectional elastomeric material. The proximal portion, in specific embodiments, is extensible in a direction that is generally parallel to the longitudinal axis of the article. Additionally, or alternatively, the proximal portion may include a releasable fold that extends in a direction transverse to the longitudinal axis of the article, with that fold being secured in place by a fastener that is configured to be selectively disengaged to allow extension of the overall dimension of the article along its longitudinal axis.

In a specific embodiment, the article includes a pull tab adjacent the first open end of the pouch and which is configured for pulling the pouch toward the base of the penis. Additionally, or alternatively, the article may include a strap adjacent to the first open end and configured for securing the pouch around the penis, with the strap including a hook and loop element that is adapted to mechanically fasten to an external surface of the pouch or the sling. The first open end of the pouch, in specific embodiments, is the only opening that is configured for insertion and removal of the penis into/from the pouch. The one or more layers of nonwoven material in the article may include a hydrophilic layer and a hydrophobic layer, with the hydrophilic and hydrophobic layers being in an overlaying relationship with one another. The pouch may include one or more stiffening elements located between the first and second ends of that pouch, with the one or more stiffening elements being configured to minimize folding of the one or more layers of nonwoven material during wear of the article. The loop portion of the sling may include a layer of lotion that is configured to lower a level of friction between the loop portion and the scrotum of the male during wearing of the article.

In another embodiment, an incontinence article is disclosed for a male having a penis and a scrotum, with that article extending along a longitudinal axis. A pouch of the article includes one or more layers of nonwoven material and is configured to envelop the penis, with the pouch having a first open end and a second closed end longitudinally opposed the first open end. The article also has a sling that is connected to the pouch adjacent the first open end, with that sling including a loop portion that is configured to fit around an underside of the scrotum and the penis, and to rest against the perineum. The loop portion is extensible in at least a generally circumferential direction of that loop portion. In that embodiment, the pouch also includes a pocket that is selectively openable to permit removal of the penis therethrough, with the pocket having an open condition in which access to an interior of the pouch is permitted, and a second condition in which that access is not permitted.

In yet another embodiment, an incontinence article is disclosed for a male having a penis and a scrotum, with the article extending along a longitudinal axis. The article includes a pouch that is configured to envelop the penis, and which includes a layer of hydrophilic nonwoven material, a layer of hydrophobic nonwoven material overlaying the layer of hydrophilic nonwoven material, and one or more stiffening elements disposed between those layers of nonwoven material. The pouch has a first open end and a second closed end longitudinally opposed the first open end, and the one or more stiffening elements minimize folding of the nonwoven layers during opening of the first open end.

The article also has a sling that is connected to the pouch adjacent the first open end of the pouch, with the sling including a loop portion that is configured to fit around an underside of the scrotum and the penis. The loop portion includes a plurality of strands oriented in a manner that makes the loop portion extensible in a generally circumferential direction of that loop portion. The article also has a pull tab adjacent the first open end of the pouch and configured to permit pulling of the pouch toward a base of the penis. A strap of the article adjacent the first open end of the pouch is configured for securing the pouch around the penis and includes a hook and/or loop element that is adapted to mechanically fasten to an external surface of the pouch or sling. Alternatively, the strap may include elastic or non-elastic strands that may be manually connected (e.g., tied or connected through a hook-and-loop fastener) to form a strap loop.

### BRIEF DESCRIPTION OF THE DRAWINGS

The objectives and features of the invention will become more readily apparent from the following detailed description taken in conjunction with the accompanying drawings in which:

FIG. 1 is a perspective view of an incontinence article deployed on the body of a male wearer, in accordance with one embodiment of the invention.

FIG. 2 is a perspective view of the incontinence article of FIG. 1 detached from the body of the male wearer.

FIG. 3 is an elevation view of the article of FIGS. 1 and 2.

FIG. 4 is an elevation view similar to FIG. 3, showing an opposite side of the incontinence article therein.

FIG. 5 is a schematic cross-sectional view taken generally along line 5-5 of FIG. 2.

FIG. 6 is a perspective view of the incontinence article of FIGS. 1-5 showing a pouch of the article in a semi-closed condition.

FIG. 7 is a view similar to FIG. 6 showing the incontinence article therein with the pouch in a relatively open condition.

FIG. 8 is a perspective view of an incontinence article in accordance with another embodiment of the invention.

FIG. 9 is an elevation view of a sling of the incontinence article of FIGS. 1-7.

FIG. 10 is an elevation view, similar to FIG. 9, illustrating a sling in accordance with another embodiment of the invention.

FIG. 11 is a perspective view of the incontinence article of FIGS. 1-7, showing temporary, pre-deployment fixation of the sling in a specific position relative to the rest of the incontinence article therein.

FIG. 12 is a perspective view illustrating application of the incontinence article of FIGS. 1-7 onto the body of a male wearer.

FIG. 13 is a perspective view similar to FIG. 12 showing another step in the application of the incontinence article therein to the body of the male wearer.

FIG. 14 is a perspective view similar to FIGS. 12 and 13 showing fixation of a strap of the incontinence article against an external surface of the article.

FIG. 15 is a perspective view similar to FIGS. 12-14 showing the incontinence article in its final, deployed position on the body of the male wearer.

FIG. 16 is a perspective view of a sling of an incontinence article in accordance with yet another embodiment of the invention.

FIG. 17 is a perspective view similar to FIG. 16 showing the sling therein in an extended condition.

FIG. 18 is a schematic view of a catheter that includes the incontinence article of FIGS. 1-7 or contemplated variations thereof.

### DETAILED DESCRIPTION OF PARTICULAR EMBODIMENTS

To the extent that any meaning or definition of a term in this written document conflicts with any meaning or definition of the term in a document incorporated by reference, the meaning or definition assigned to the term in this written document shall govern. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless specified or limited otherwise, the terms “mounted,” “connected,” “supported,” and “coupled” and variations thereof are used broadly and encompass both direct and indirect mountings, connections, supports, and couplings.

Further, “connected” and “coupled” are not restricted to physical or mechanical connections or couplings. Also, as used herein, the term “releasable coupling,” “releasable attachment,” and related terms refer to a type of coupling/attachment in which the coupled/attached structures may be readily detached, decoupled, or otherwise separated from one another in a simple manner and without causing the destruction or damage of any of those structures.

For the sake of further explanation, a permanent-rather than a “releasable”-type of coupling/attachment/connection may refer, for example, to two structures that are integrally formed with one another, or which are adhesively attached with some type of non-releasable adhesive, such that their separation would necessarily result in at least some level of damage to one or more of the parts being separated from one another. For ease of explanation, and unless otherwise stated, identical numerals throughout this description and figures refer to similar features in the various embodiments herein described, with the structure and functionality of those features as described with respect to a particular embodiment being similarly applicable to the identically numbered features in other embodiments. It should also be noted that similar features in various embodiments may be identified by different numerals to facilitate understanding of the embodiment being described, notwithstanding their similarity in structure and/or functionality.

It should also be noted that, as used herein, the term “elastomeric” refers to a material that, when subjected to a stretching force, is stretchable in the direction of that stretching force, and which upon release of that stretching force returns to approximately its original, relaxed dimension. Also, as used herein, the term “extensible” and derivatives thereof refer generally to a material that stretches in the direction of an applied stretching force by at least about 25% of its relaxed dimension and which returns to approximately its original, relaxed dimension upon release of that stretching force.

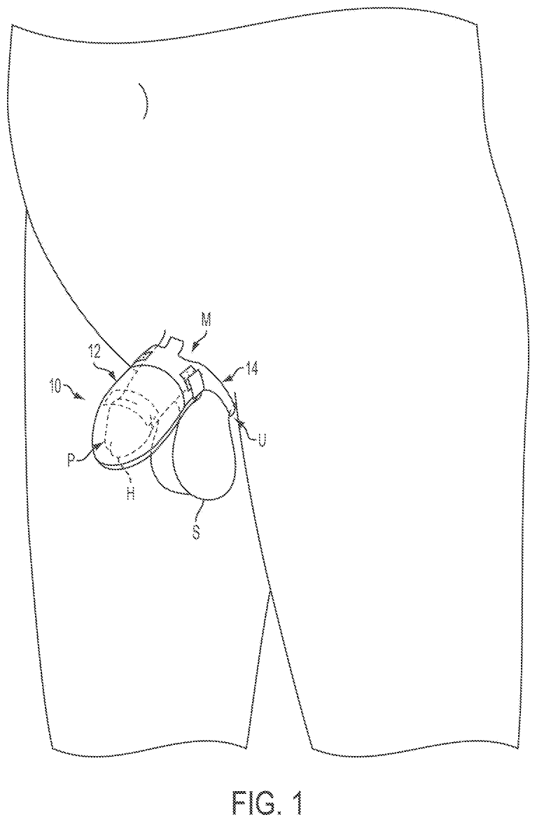

With reference to the figures, and particularly to FIGS. 1, 2, 3, and 4, an illustrative embodiment of a male incontinent article 10 includes features to comfortably envelop the wearer's penis P in a manner that provides suitable levels of urine containment while being firmly supported in place. The embodiment shown in those figures is a disposable incontinent product, although it is contemplated that it may instead have components that are reusable, as explained in further detail below. Article 10 includes two main components: a pouch 12 and a sling 14 connected to pouch 12 and configured to support article 10 anchored to an underside U of the scrotum S and an oppositely disposed area M at the base of the wearer's penis P.

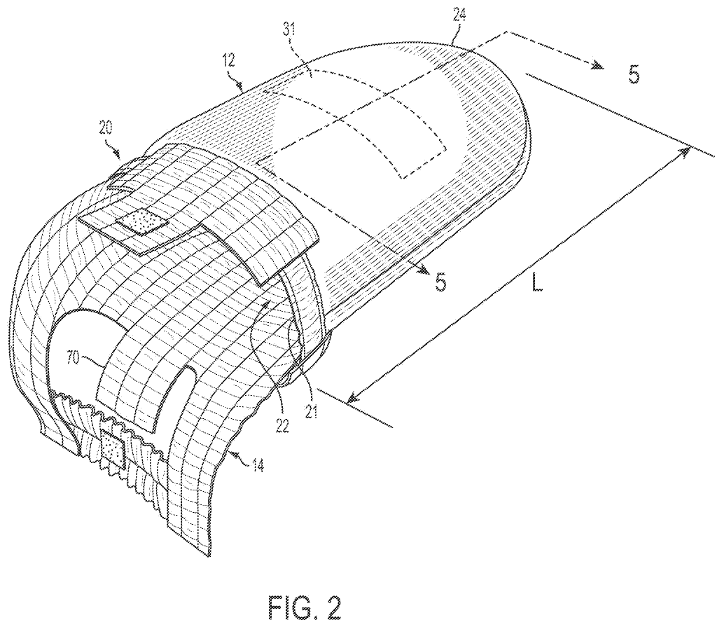

Pouch 12 of the example embodiment of FIGS. 1-4 is a generally conical, frustoconical, or tongue-shaped hollow structure, having a length dimension L (FIG. 2) that extends along a longitudinal axis X of article 10. Pouch 12 has a first, open end 20 defining an opening 21 that allows insertion of penis P into an interior 22 of pouch 12, and a longitudinally opposed second, closed end 24. During wear, the head H of the penis P is adjacent closed end 24, while first, open end 20 is disposed relatively closer to the base of penis P, as seen in FIG. 1.

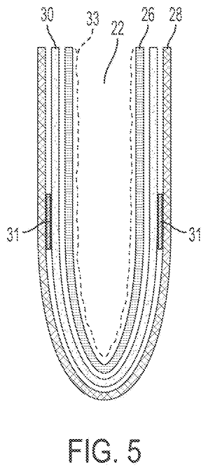

With continued reference to FIGS. 1-4, and further referring to FIG. 5, pouch 12 may include one or more layers of nonwoven material, which permit air flow to occur between interior 22 and the exterior. FIG. 5 illustrates, by way of a non-limiting example, a structure that may be suitable for pouch 12. The example pouch 12 in that embodiment includes an inner layer 26 of hydrophilic nonwoven material configured to be in direct contact with penis P, an outer layer 28 of hydrophobic nonwoven material defining an outer surface of article 10, and an absorbent core 30 disposed between layers 26 and 28. The hydrophilic nature of layer 26 readily allows the flow of leaked urine into an interior of the layered structure, and more specifically into absorbent core 30.

Conversely, the hydrophobic nature of layer 28 prevents or at least minimizes the flow of leaked urine (or other fluids) to the exterior of article 10, thereby minimizing the likelihood that the leaked urine will wet the underwear and/or pants of the wearer. While not shown, article 10 may further include an ADL between inner layer 26 and absorbent core 30, to absorb and distribute leaked urine toward absorbent core 30 more effectively and rapidly.

Notably, the nonwoven materials making up pouch 12 allow the flow of air between interior 22 and the exterior, which provides for enhanced comfort for the wearer e.g., minimizing the accumulation of sweat. Further, the nonwoven-based construction of pouch 12 minimizes the embarrassing noise otherwise made by a hypothetical pouch made of hard plastic films or the like. It should be noted, however, that the flow of air facilitated by the non-woven based construction of pouch 12 may be detrimental in other respects. Specifically, that type of construction may result in urine or other odors to readily flow from interior 22 toward the exterior, which may be embarrassing and uncomfortable to the wearer. Alternative embodiments are contemplated in which the material or materials making up pouch 12 are a woven or textile material, such as one made up primarily of cotton or blends of cotton or some other material(s) that allow(s) the flow of air as described above.

The nonwoven materials making up the structure of pouch 12 may be the same or may be different from one another. In a specific embodiment, the outer layer 28 of pouch 12 may be in the form of a material known in the trade as Sawabond® 7011, a carded nonwoven having a basis weight of about 22 grams/m² (“gsm”), treated for hydrophobicity, commercially available from the Sandler Group of Schwarzenbach/Saale, Germany. Another suitable nonwoven material is a 15 gsm material known under the trade name Elite Soft® (treated for hydrophobicity) commercially available from Fibertex Nonwovens, of Aalborg, Denmark. Yet other suitable materials are the 15 gsm or 17 gsm versions of a spunbond polypropylene CD-rod bond nonwoven material, treated for hydrophobicity, known in the trade as SPL365398-99, commercially available from Fitesa of High Point, North Carolina.

A material suitable for the inner layer 26 of pouch 12 is known in the trade as Sawabond® 4357, a nonwoven having a basis weight of about 18 gsm, treated to be hydrophilic, or one identified in the trade as Sawabond® 7060, a nonwoven having a basis weight of about 22 gsm, treated to be hydrophilic. Both of these example materials are commercially available from the Sandler Group of Schwarzenbach/Saale, Germany. Another suitable material for the inner layer 26 is a material known in the trade as DWE 556KW-30 12DA, a spunbond polypropylene CD-rod bond material having a basis weight of about 12 gsm and commercially available from Fitesa, of High Point, North Carolina. Yet another suitable material is the 15 gsm Elite Soft® material cited above (in connection with outer layer 28) but treated instead to be hydrophilic.

As stated above, the structure making up pouch 12 includes an absorbent core 30 disposed between the inner and outer layers 26, 28. In a specific embodiment, the absorbent core 30 is free of fluff pulp, which results in an absorbent core 30 that is relatively thin. In other embodiments, the absorbent core 30 has a relatively small amount of fluff pulp, for example up to about 25% by weight and more specifically in particular embodiments about 10% to 15% by weight, thereby resulting in a relatively thin absorbent core compared to a hypothetical core having—for example—greater than about 50% by weight of fluff pulp. Thinness may be advantageous to enhance the wearing experience of article 10, specifically making the article less cumbersome and bulky to wear under any briefs and/or outer pants worn by the man. Notwithstanding, the perceived thinness of article 10 resulting from a fluff pulp-free absorbent core may give the wearer and/or caregiver the false impression that article 10 is not effective at absorbing leaked urine at required levels, which may be detrimental to the overall commercial appeal of article 10.

A suitable material for absorbent core 30 is a 135 gsm (basis weight) version of an absorbent material known in the trade as TLA® and commercially available from Fitesa, of High Point, North Carolina. That material includes about 28% by weight of SAP, an acquisition layer, a small amount of fluff pulp (about 10% to about 15%), and a storage layer-all built into and commercially available as an integrated laminate. The presence of an integrated acquisition layer in this material does not necessarily obviate the desire, in particular embodiments, to have a separate ADL in the construction of pouch 12, located between inner layer 26 and absorbent core 30. Yet another suitable material option for absorbent core 30 is an environmentally-friendly biopolymer SAP known in the trade as azuraGel®, commercially available from Ecovia Renewables, Inc. of Ann Arbor, Michigan. In embodiments using a pouch 12 with materials of the type described above, the resulting thickness of the pouch is about one seventh the thickness of a conventional diaper, and about one third the thickness of a conventional incontinence pad, which makes article 10 a more sustainable incontinence article than diapers and pads.

In embodiments that incorporate an ADL between the inner layer 26 and the absorbent core 30, the ADL may have ridges and resulting channels defined by those ridges. Those ridges and resulting channels may help direct the flow of urine or other secretions and restrict its flow toward opening 21 of pouch 12, thereby minimizing the likelihood of undesirable leakage out of pouch 12. Those ridges may, for example, be oriented transversely to axis X of article 10, although other orientations are contemplated as well. Additionally or alternatively, inner layer 26 may have ridges and resulting channels to similarly direct the flow of urine or other secretions and restrict its flow toward opening 21 of pouch 12.

Notably, ridges in the inner layer 26 may minimize the total amount of surface area of inner layer 26 that is available for direct contact with penis P, which enhances comfort during wear. For example, ridges in the inner layer 26 may be of dimensions and spacings between them that is effective to reduce the amount of surface area of inner layer 26 by at least about 20% relative to an inner layer 26 that is essentially flat. Depending on the dimensions and spacings between ridges, that reduction of surface area may be at least about 40%, at least about 50%, or even at least about 70% relative to an inner layer 26 that is essentially flat. For embodiments including an ADL, ridges in that material, if the inner layer 26 conforms to the ADL, may similarly reduce the amount of surface area of inner layer 26 that is available for direct contact with penis P, which enhances comfort during wear.

It should be noted, notwithstanding, that ridges in the inner layer 26 and/or ADL may cause urine or other secretions not to be evenly distributed across the absorbent feature of pouch 12 (e.g., absorbent core 30), limiting absorption only to areas under the channels defined by the ridges, thereby decreasing the overall absorption capacity of pouch 12. That decrease in overall absorption capacity may, in turn, lead to undesired leakage or the need to replace article 10 more often than would otherwise be necessary.

Other suitable materials for absorbent core 30 are the 163 gsm, 170 gsm, 200 gsm, and 250 gsm (all nominal basis weight values) versions of TLA®, with higher basis weights (while keeping the same dimensions in article 10) resulting in higher absorbent capacity. Other suitable materials for absorbent core 30 include one known in the trade as TotalCore®, a fluff pulp-free airlaid material having a basis weight of about 186 gsm and commercially available from Gelok International Corporation, of Dunbridge, Ohio, and an absorbent material known under the trade name Amphibia®, commercially available from the TWE Group, of Emsdetten, Germany, which is a fluff pulp-free and adhesive-free multilayer substrate that includes SAP.

The dimensions, type, and basis weight of the materials making up absorbent core 30 are suitably chosen such that article 10 has an absorbent capacity for urine of at least about 85 ml, and more specifically about 100 ml and in particular embodiments about 150 ml. For example, and without limitation, article 10 may include an absorbent core made from the 135 gsm TLA®, and about 28% SAP, with chosen dimensions yielding a total area of about 26.1 in² (about 180.6 cm²), for a resulting total absorption capacity of about 85 ml. Another example using the same dimensions but made from the 170 gsm version of the TLA® material (with about 28% SAP) results in a total absorption capacity of about 100 ml.

Yet another example using the same dimensions but made from the 250 gsm version of the TLA® material (with about 28% SAP and about 20-25% fluff pulp by weight) results in a total absorption capacity of about 150 ml. The same construction as discussed above but using instead a 200 gsm version of the TLA® material (with about 28% SAP and about 20-25% fluff pulp by weight) results in a total absorption capacity of about 115 ml, while one using a 300 gsm version of the TLA® material (with about 28% SAP and about 20-25% fluff pulp by weight) results in a total absorption capacity of about 200 ml. The respective thicknesses and other properties of the pouch 12 when using the materials discussed above are summarized in Table 1, below:

TABLE 1
Pouch Thickness for each Absorbent Core material

| | | | | Approx. | |
|---|---|---|---|---|---|
| | | | Area of | Resulting | Resulting |
| | SAP | Approx. Fluff | absorbent | Absorbing | Pouch |
| Core | Content | Pulp content | core in in² | Capacity of | thickness |
| Material | by weight | by weight | (cm²) | Pouch (ml) | (mm) |
| 135 gsm | 28% | 10-15% | 26.1 (180.6) | 85 | 2 |
| 170 gsm | 28% | 10-15% | 26.1 (180.6) | 100 | 2.5 |
| 200 gsm | 28% | 20-25% | 26.1 (180.6) | 115 | 4 |
| 250 gsm | 28% | 20-25% | 26.1 (180.6) | 150 | 5 |
| 300 gsm | 28% | 20-25% | 26.1 (180.6) | 200 | 6.5 |

With continued reference to FIGS. 1-5, it is contemplated that the dimensions of layers 26, 28, and of absorbent core 30 may be all the same as one another, or they may be different. For example, absorbent core 30 may be smaller (in the planar dimension) than both layers 26, 28, to minimize the absorbent capacity of article 10 and/or to make article 10 less bulky than necessary. In the example of embodiment of FIGS. 1-5, the opening 21 at the first end 20 of pouch 12 provides the only opening of pouch 12 configured to permit insertion and/or removal of penis P from the interior 22, which minimizes the likelihood of undesirable leakage through a hypothetical slot or other large, exposed opening in the body of pouch 12. Notably, the continuous enclosure provided by the example pouch 12 in those figures thus results in the need to remove penis P from article 10 for the wearer to urinate at will, for example, in a bathroom, which may be inconvenient to the wearer and/or his caregiver.

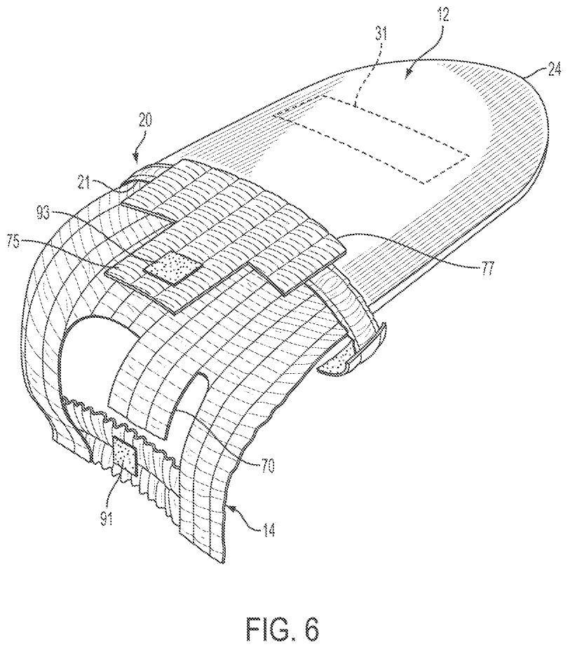

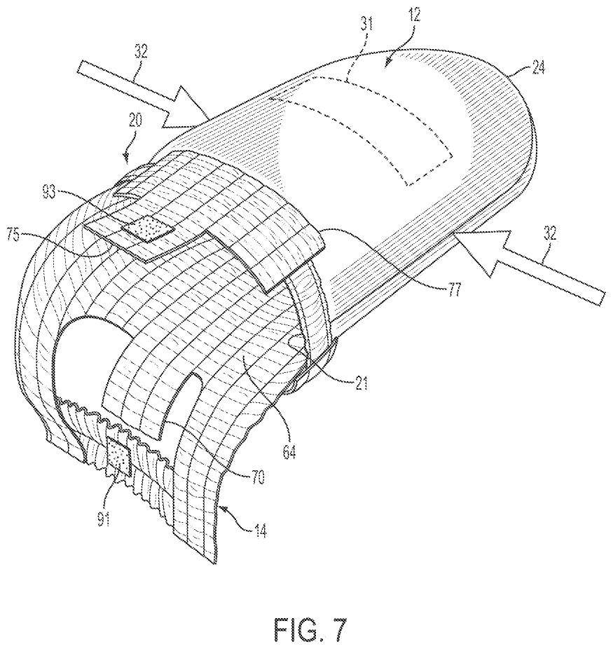

With continued reference to FIGS. 1-5 and further referring to FIGS. 6 and 7, the pouch 12 includes one or more stiffening elements 31 disposed between inner and outer layers 26, 28. Stiffening element 31, which in the illustrated embodiment has a generally rectangular shape, prevents or at least hinders the folding of the nonwoven layers of pouch 12 during application of article 10 i.e., during insertion of penis P into interior 22. FIGS. 6 and 7 show the effect of the presence of stiffening element 31 during such application. Specifically, as the wearer or caregiver exerts inward forces against the sides of pouch 12 (arrows 32 in FIG. 7), the opening 21 at first end 20 naturally expands rather than collapsing, by virtue of the bending resistance of stiffening element 31. This facilitates application of the article 10 (i.e., insertion of penis P into interior 22 of pouch 12), particularly making that a single-handed operation.

Stiffening element 31 is made of a material that has a greater rigidity/bending resistance (i.e., it has a higher flexural modulus) than the material or material combination making up the body of pouch 12, particularly in the transverse dimension of the article 10 i.e., transverse to longitudinal axis X. It is contemplated, in that regard, the stiffening element 31 may have the same or at least substantially the same flexural modulus in the transverse dimension, relative to the longitudinal dimension (i.e., along axis X) of article 10. Suitable materials making up stiffening element 31 include, without limitation, a Thermoplastic Polyurethane (“TPU”) based material known under the trade name Desmopan®, specifically one such material in the Product series 3, Product series 4, or Product series 9, all available commercially from Covestro AG, of Leverkusen, Germany.

Those example materials have a flexural modulus, using the standardized test known as ISO 178 (“Plastics-Determination of Flexural Properties”) at a test setting of 2 mm/min, of at least 110 Mpa. Other suitable materials include chloroprene rubber Grade 65A (“Neoprene”) known in the trade under the designation DRC-20, commercially available from Delta Rubber Ltd. of Dorset, United Kingdom, silicone semi-transparent white Grade P60, commercially available also from Delta Rubber Ltd., and a 0.5 mm thick material known in the trade as Dritz® woven non-roll elastic (designation: 9407W), which is a substrate made of 70% polyester and 30% rubber available from Prym Consumer USA Inc., of Spartanburg, South Carolina.

Some of the above-named sample materials for stiffening element 31 have a tendency to more or less retain a bowed shape upon exertion of lateral forces (arrows 32 in FIG. 7) sufficient to cause the opening 21 of pouch 12 to expand for insertion of penis P. The retention of the bowed shape has been found to be beneficial in that it prevents the pouch 12 of article 10 from being biased toward a flat position (i.e., a closed position of opening 21), thereby preventing uncomfortable pressing of the interior surfaces of pouch 12 against penis P during use. It should be noted, however, that retention of a bowed shape as explained above may be detrimental to the overall wearing experience insofar as the wearer and/or caregiver may perceive article 10 to be permanently deformed upon the exertion of lateral forces (arrows 32 in FIG. 7) to cause opening 21 to expand. Additionally, there may be wearers that would prefer the interior surfaces of pouch 12 to press against penis P in use for a sensation of secure fitting of article 10. In that regard, having a stiffening element 31 that retains a bowed shape would be detrimental to the wearing experience for those users.

The inventors have found that a stiffening element 31 made of a material having a flexural modulus of at least 80 Mpa (at least in the transverse dimension of article 10), and specifically 110 Mpa and more specifically—in particular embodiments—of between about 110 MPa and about 230 Mpa-creates a sufficient difference in rigidity (bending resistance) relative to the material(s) making up the rest of pouch 12 that allows stiffening element 31 to function as described herein. It should be noted, notwithstanding, that a flexural level greater than 80 Mpa for the stiffening element 31 may cause discomfort to the wearer the article 10, or unduly make the entirety of article 10 less flexible than desired by the wearer and/or make article 10 relatively bulky, which may be detrimental to the overall wearing experience and commercial appeal of article 10.

Each stiffening element 31 in the embodiment of FIGS. 1-7 is generally rectangular, therefore including straight edges and relatively sharp corners. That is advantageous from a manufacturing standpoint, since many such stiffening elements 31 can be made from a strip of material having the final desired length of that element and cut at predetermined intervals (to yield the final width of the stiffening element 31). However, it should be noted that straight edges and/or relative sharp corners of stiffening element 31 may make article 10 visually and tactfully less appealing to the wearer than a shape that has rounded edges and no sharp corners. It is contemplated, in that regard, that stiffening element 31 could have any shape other than the one shown in the figures.

Further, while the embodiment in FIG. 5 includes two diametrically opposed stiffening elements 31, embodiments are contemplated in which pouch 12 includes only one stiffening element 31 or any number of stiffening elements having shapes and/or structures that are similar or different from one another. The presence of two stiffening elements 31 in the embodiment of FIG. 5 further facilitates expansion of the opening 21 at first end 20 upon inward exertion of forces on the sides of pouch 12 (arrows 32 in FIG. 7), relative to an embodiment having a single stiffening element 31. In that regard, it may be disadvantageous for a pouch 12 to have only one stiffening element 31, due to the probability that opening 21 will not expand as desired upon the exertion of forces (arrows 32).

The absence of any adhesive, elastic bands or other means for directly securing pouch 12 to penis P provides the wearer of article 10 with enhanced comfort, allowing the penis to move and go through the natural cycles of erection during sleep. FIG. 5 illustrates an optional feature that further enhances the wearer's comfort. Specifically, the embodiment in that figure includes a layer 33 of a skin-care composition, such as one containing petrolatum, on the interior-facing surface of inner layer 26, that effectively lowers the level of friction against the penis P during wear. The skin-care composition may be suitably chosen to provide a medical benefit, such as medicated ointments applied upon the occurrence of injury or post-surgery. In that regard, the skin-care composition used in article 10 may be such that it enhances healing of penis P or surrounding areas when necessary. While FIG. 5 shows layer 33 as a discontinuous pattern, it is contemplated that same could instead be continuous or be discontinuous and in any chosen pattern, uniform or non-uniform, such as one including stripes, dots, geometric figures, or any random shape, repeated or not repeated, and with any suitably chosen density.

The skin-care composition defining layer 33 may be solid or semi-solid at 20° C. and may be applied to yield a basis weight in the range between about 1 and about 40 gsm, for example, specifically between about 2 gsm and about 16 gsm in some embodiments, and more specifically between about 4 gsm and about 10 gsm in other embodiments. Suitable types of skin-care compositions are provided in U.S. Pat. No. 6,270,487 to Sheehan et al., hereby incorporated by reference herein solely for purposes of providing examples of such compositions, and solely incorporating those portions of that patent containing such disclosure.

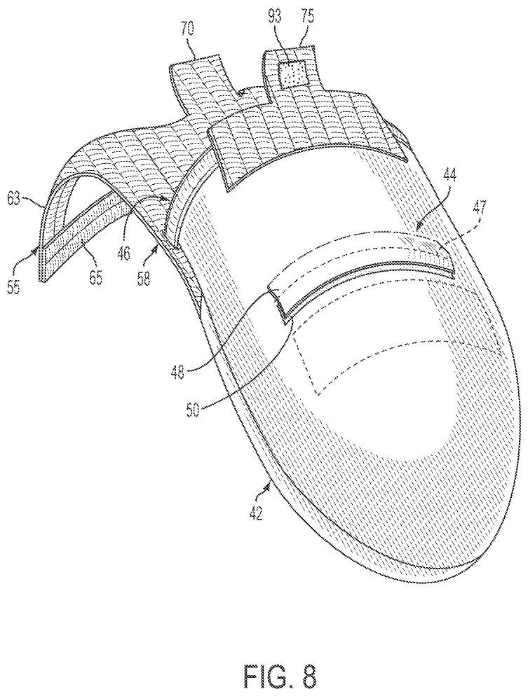

FIG. 8 illustrates an alternative embodiment of a pouch 42 having a selectively openable and closeable pocket 44 that, in the open condition of that pocket 44, allows the wearer or his caregiver to selectively remove the penis P from an interior 46 of pouch 42 with relatively ease, obviating the need to remove/disengage the entire article from penis P. Pocket 44 includes a secondary opening 47 large enough to permit a portion of the penis P to exit the interior 46, and a flap 48 that selectively and releasably closes the secondary opening 47. To that end, a releasable fastener 50, such as adhesive tape, a hook-and-loop fastener, or a snap fastener, secures the flap 48 relative to the rest of pouch 42 to provide a closed condition of that pocket 44.

In use, upon conclusion of the event that necessitated the temporary removal of a portion of the penis P (at least the distal end portion of penis P that includes the head H), the penis P is returned fully into interior 46 of pouch 42 and the releasable fastener 50 made to secure flap 48 to the rest of pouch 42. Notably, the discontinuity in the body of pouch 42 provided by secondary opening 47—for example compared to the continuous body of the pouch 12 in the preceding figures—may lead to undesirable leakage. But this is a feature that nonetheless may enhance the overall experience of the wearer, particularly one that would prefer not to have to disengage/disconnect the entire article from his body, particularly making using the restroom quicker, easier, and more convenient.

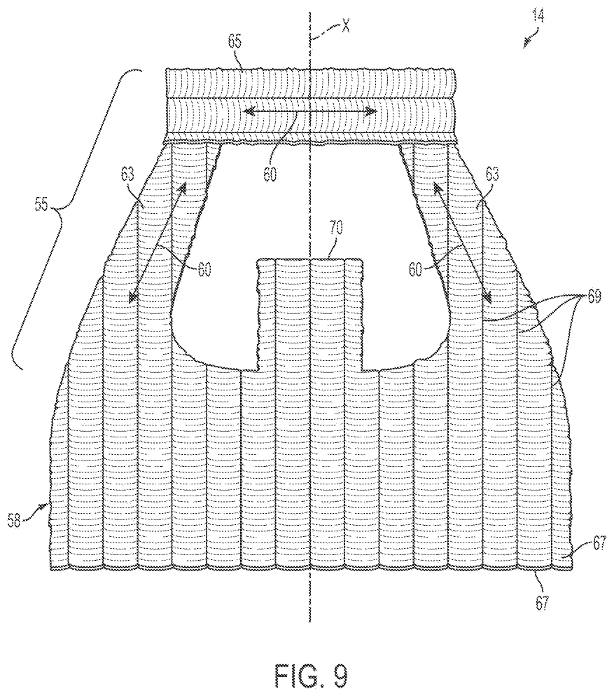

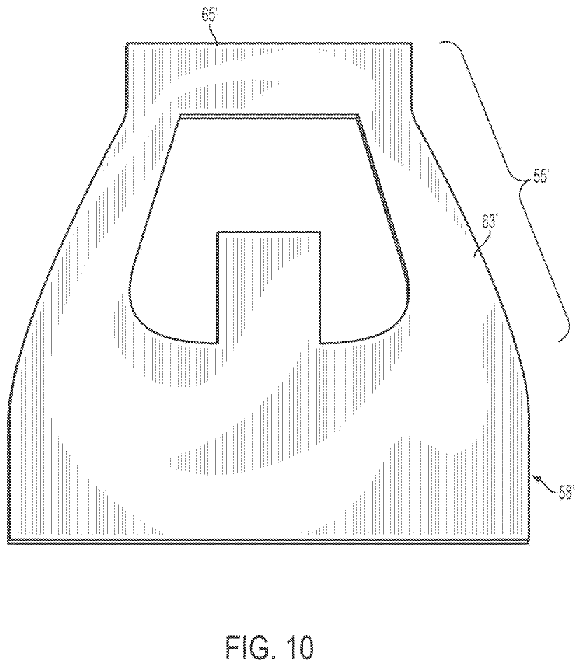

With continued reference to FIGS. 1-8 and additionally referring to FIGS. 9 and 10, the various features of sling 14 are illustrated. Sling 14, as noted above, is configured to comfortably support the weight of article 10, anchored to an underside U of the scrotum S and to an oppositely disposed area M at the base of the wearer's penis P (FIG. 1). To that end, sling 14 is an elasticized structure connected to and extending from the area adjacent first, open end 20 of pouch 12, with the elasticity of sling 14 providing for comfortable and secure engagement of article 10, particularly to underside U of scrotum S. Referring with particularity to FIG. 9, sling 14 includes a loop portion 55 configured to fit around scrotum S and to lie against underside U during wear, and a proximal portion 58 that is connected to pouch 12. In specific embodiments, proximal portion 58 is integrally formed with pouch 12. In the illustrated example embodiment of FIG. 9, proximal portion 58 is a separate structure that is coupled to pouch 12 in an area adjacent first, open end 20.

Loop portion 55 of sling 14 is extensible at least in the generally circumferential direction of loop portion 55 (arrows 60). As used herein, the term “circumferential” is not intended to imply that loop portion 55 is necessarily circular or forms part of a circular structure, but rather is used to describe the general direction along the length of the one or more components making up loop portion 55. In the embodiment of the figures, loop portion 55 is generally horseshoe-shaped and made up of two leg portions 63 extending from and connected to the proximal portion 58, and a distal portion 65 connecting the distal, terminal ends of leg portions 63.

Each of the leg portions 63 is made of a pair of layers 67 of nonwoven material overlying one another, and a plurality of elastic strands 69 disposed between layers 67. In their natural, at-rest state, the elastic strands 69 are unstretched and attached (e.g., adhesively) to layers 67, with portions of the material making up layers 67 in a “gathered” state, forming ripples in that material. The functional result of that construction is that, upon the exertion of a pulling force acting on the ends of a leg portion 63, the elastic strands 69 and gathered nonwoven material of layers 67 extend together, thereby making that leg portion 63 extensible in the direction of the pulling force (arrows 60). The strands 69 of each leg portion 63 are oriented, in the illustrated embodiment, in a direction generally parallel to the longitudinal axis X of article 10 but they nevertheless provide extensibility in the direction of arrows 60, which is angles to axis X, as seen in FIG. 9. In other contemplated embodiments, the orientation of the strands 59 of leg portions 63 follows the arched shape of the leg portions 63, rather than being generally parallel to longitudinal axis X.

A material suitable for one or both of the layers 67 making up leg portions 63 is a 24 gsm, hydrophobically treated nonwoven material known under the trade name Sontara® commercially available from Jacob Holm of Basel, Switzerland. Regarding the elastic strands forming part of the leg portions 63, a suitable type of elastic strand material is one known under the trade name Hyfit®, commercially available from the Lyca Company of Wilmington, Delaware. That elastic strand material has a decitex value of 1240. Another material suitable for leg portions 63 is a cotton, polyester, and rubber blend compression gauze/bandage known in the trade as Ace®, commercially available from the 3M Co. of St. Paul, Minnesota.

In the example embodiment of FIGS. 1-9, the leg portions 63 have an arcuate or arched shape, bending inwardly toward distal portion 65. Further in that embodiment, distal portion 65 is a separate structure having a generally elongated, rectangular shape, that is coupled to the distal, terminal ends of leg portions 63 through ultrasonics, adhesive, or some other known method for securing nonwoven-based structures to one another. It is contemplated that the shape of distal portion 65 may be different from that shown in the figures. For example, distal portion 65 may be arcuate so as to complement the arched shape of leg portions 63, thereby making the entire loop portion 55 generally closer to circular or at least closer to being horseshoe shaped. Yet other contemplated shapes of distal portion 65 and/or leg portions 63 include end areas of distal portion 65 and/or of each leg portion 63 that may be narrower than the respective central areas of those portions, thereby resulting in a loop portion 55 that more closely conforms to the anatomical shapes in the wearer's genitalia.

Distal portion 65 in the illustrated embodiment has a construction similar to that of leg portions 63, and in that regard, is made up of a pair of layers 67 of nonwoven material overlying one another, and a plurality of elastic strands 69 disposed between layers 67. Notably, the elastic strands 69 in distal portion 65 are oriented transversely to the longitudinal axis X of article 10 and to the orientation of strands 69 in leg portions 63. That orientation makes distal portion 65 extensible in a direction transverse to longitudinal axis X, which complements the direction of extensibility of leg portions 63, thereby making the entirety of loop portion 55 extensible in the generally circumferential direction of loop portion 55 (arrows 60). Notably, the extensibility of distal portion 65 in a transverse direction (i.e., transverse to longitudinal axis X) allows loop portion 55 of sling 14 to sit flat across the natural curved shape of the wearer's perineum. Further, that transverse extensibility makes it easier for the wearer and/or caregiver to maneuver loop portion 55, and slip same over the scrotum during application and secure same against underside U, all without twisting of loop portion 55. Once securely engaged to underside U, the retraction of the extensible parts of sling 14 causes the entire article 10 to be securely held in place against the wearer's genitalia.

FIG. 10 illustrates an alternative embodiment of a structure defining a loop portion 55′ and proximal portion 58′ of a sling. In that embodiment, proximal portion 58′, leg portions 63′ and distal portion 65′ are all made of a material with multidirectional extensibility, thus obviating the need for any elastic strands 69. Further, in that example embodiment, the proximal portion 58′, leg portions 63′, and distal portion 65′ are formed integrally with one another, rather than being separate components secured together through adhesive, ultrasonics, or some other means.

A suitable multidirectionally extensible material that may be used as described above is a material known under the trade name Conforma® 9800, commercially available from HB Fuller of Saint Paul, Minnesota. That material has a nominal basis weight of about 100 gsm and a thickness of about 1 mm, being a single layer structure made up of a mixture of fiber and elastomeric adhesive and capable of stretching by about 300% of its initial dimensions. Another material suitable for this purpose is a dual layer structure that includes a layer of 25 gsm spunlace nonwoven and 75 gsm elastomeric adhesive. Other materials that are also suitable for this purpose are three-layer structures made up of an elastomeric material sandwiched between two overlying layers of nonwoven, such as those known in the trade as Elstipro® (commercially available from Berry Global of Evansville, Indiana) and SoftFit® (commercially available from Aplix of Le Cellier, France). Yet another suitable material is known in the trade under the name Rebound®, which is a breathable webbed elastomeric material sandwiched between nonwoven layers, commercially available from Mativ, of Alpharetta, Georgia.

Other embodiments are similarly contemplated for the structure making up loop portion 55, 55′ in any of the embodiments in the preceding figures. For example, loop portion 55, 55′ may be made-up of the multidirectionally elastomeric, extensible material described above, but in three separate components i.e., each of the leg portions 63, 63′ and distal portion 65, 65′ being separate components that are coupled together. Yet other embodiments that are contemplated include a loop portion similar to that of FIGS. 1-9 but with elastic strands 69 in a number other than what is shown in those figures. For example, one or more of the leg portions 63 and distal portion 65 may include a single elastic strand 69 rather than a plurality of elastic strands. Embodiments are also contemplated in which leg portions 63 and distal portion 65 include additional elastic strands oriented transversely to the strands 69 in the embodiment of FIGS. 1-9, thereby giving leg portions 63 and distal portion 65 multidirectional extensibility. It is also contemplated that one or more of the leg portions 63 and/or distal portion 65 may be made up of a pair of overlying nonwoven layers and a multidirectionally extensible mesh material disposed between or otherwise adjacent those layers.

With continued reference to FIGS. 1-9, proximal portion 58 of sling 14 may have a structure similar to or different from any of the illustrated constructions and contemplated variations of the leg portions 63 or distal portion 65 in that embodiment. For example, in the example embodiment of FIG. 9, proximal portion 58 has the same multilayer construction as that of leg portions 63 and distal portion 65. Notably, however, the orientation of the strands 69 making up proximal portion 58 is such that proximal portion 58 is extensible in a direction generally parallel to, rather transverse to, the longitudinal axis X of article 10. Specifically, in that illustrated embodiment, the orientation of the strands 69 in proximal portion 58 is similar to that of the strands 69 making up leg portions 63. Further, in that example embodiment, the leg portions 63 and proximal portion 58 are integral with one another, and made as a unitary substrate, from the same material. Yet other embodiments are contemplated in which proximal portion 58 has no extensibility in any direction, while leg portions 63 and/or distal portion 65 are extensible, or in which the levels and/or direction of extensibility between one or more of proximal portion 58, leg portions 63, and distal portion 65 differ from one another.

Proximal portion 58 in the illustrated embodiment is only partially connected to pouch 12, thereby leaving an unattached portion 64 (FIG. 4) of that substrate to move independently of pouch 12. That freedom of movement of the unattached portion 64 of proximal portion 58, along with the longitudinal direction of extensibility of that unattached portion 64 (in the general direction of axis X), provides for added comfort to the wearer of article 10. Specifically, that feature allows movement of article 10 during wear in the longitudinal direction (i.e., generally along axis X), accommodating longitudinal movement of the penis P, as well as gravity-based movement of article 10 resulting from the weight of the leaked urine absorbed by pouch 12. While not shown, it is contemplated that sling 14 may include a layer of skin-care composition similar to the layer 33 of skin-care composition described above with respect to pouch 12.

The inventors have found that, in use, the sling 14 of article 10 is normally stretched in the longitudinal direction (i.e., generally along axis X) by about 20% to about 30%, and more specifically between about 28% and about 29%. In that regard, the inventors have further found that the material or material combination making up sling 14 may be chosen such that the retraction force (in the longitudinal direction) of sling 14 in the about 20 to 30% strain range does not exceed about 1.3 N. That level of retraction force in use strikes the right balance between comfort to the wearer (which makes low retraction forces desirable) and securely maintaining article 10 in place during normal wear (which makes high retraction forces desirable).

The measurement discussed above is obtained following a standard force vs. strain (displacement) tensile test, which utilizes an Instron® machine to stretch out the entire sling 14 in the longitudinal direction X. It should be noted, notwithstanding, that the relatively low level of retraction forces (such as those no greater than about 1.3 N) may be detrimental to the wearer's sense of security, leading him to believe that the article 10 will not stay securely in place during wear. Table 2, below, shows the average retraction forces for three different materials or material combinations that were found suitable for sling 14.

TABLE 2
Average Retraction Force of Sling at
different strain levels (in Newtons)

| Sling | 20% | 25% | 30% |
|---|---|---|---|
| Construction | Strain | Strain | Strain |
| A | 0.52 | 0.71 | 0.88 |
| B | 0.49 | 0.62 | 0.75 |
| C | 0.79 | 1.03 | 1.27 |

In Table 2, above, sling construction A is the material discussed above known in the trade as Conforma® 9800, available commercially from HB Fuller. Sling construction B is made up of a plurality of T837 Hylift Elastane® elastic strands of 1240 decitex value and 5 mm spacing between strands, adhesively secured to and sandwiched between two layers of Sontana® 24 gsm hydrophobically-treated nonwoven layers. Sling construction C is similar to sling construction B, except that it had a combination of T837 Hylift Elastane® elastic strands of 470, 620, and 800 decitex values and a spacing between strands ranging between 5.1 mm and 8.5 mm. For sling constructions B and C, above, the Sontana® 24 gsm hydrophobically-treated nonwoven is commercially available from Jacob Holm & Sons AG of Basel, Switzerland (now part of the Glatfelter Corp. of Charlotte, North Carolina) and the T837 Hylift Elastane® elastic strands are commercially available from the Lycra Co. of Wilmington, Delaware.

The example embodiment FIGS. 1-9 also includes a pull tab 70 extending longitudinally from proximal portion 58 and made of the same material as that of proximal portion 58, thereby rendering the pull tab 70 also extensible in the longitudinal direction of article 10. When applying the product to the penis P, the wearer pulls on the pull tab 70 to more easily move article 10 towards the base of the penis P. In the illustrated embodiment, article 10 includes a second pull tab 75, disposed opposite pull tab 70 and made of the same material defining proximal portion 58.

In that example embodiment, the second pull tab 75 is formed from a T-shaped substrate 77 attached to an area of the first, open end 20 diametrically opposite from proximal portion 58 of sling 14. Some wearers and/or their caregivers may find having two pull tabs 70, 75 rather than a single one preferable to further facilitate pulling of article 10 toward the base of penis P during application. In the illustrated embodiment, the second pull tab 75, being made of the same material as that defining proximal portion 58, is similarly extensible in a direction generally parallel to the longitudinal axis X of article 10.

It is contemplated that article 10 may alternatively have no pull tabs at all or have pull tabs in a number other than the two in the illustrated embodiment. Further, it should be noted that pull tabs 70, 75 may have shapes or constructions that are different from one another or have shapes and/or constructions that differ from those described above and illustrated in the figures or be located in areas of article 10 that differ from those shown in the figures.

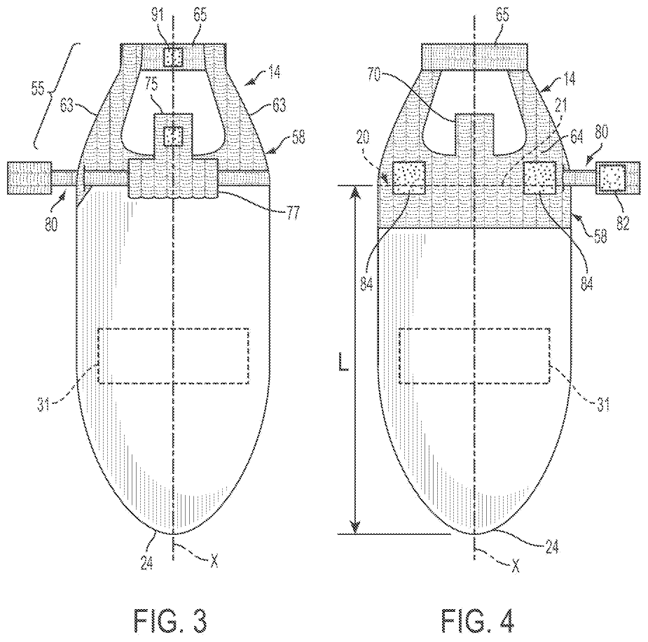

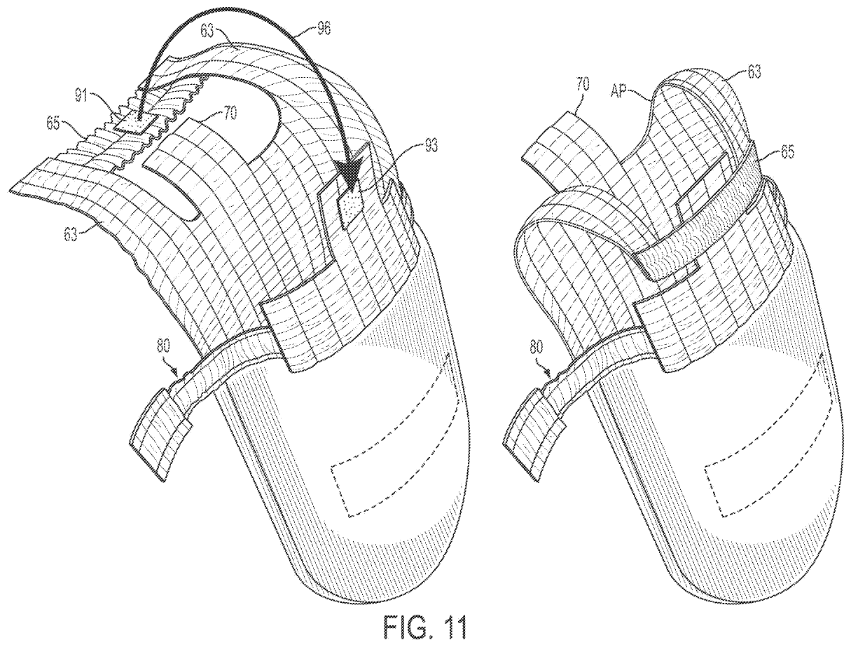

Referring particularly to FIGS. 3, 4 and referring additionally to FIG. 11, article 10 also includes a strap feature that aids in securing article 10 against the body of the wearer. That strap feature is made-up of an elongate, elasticized, extensible strap 80 extending transversely to the longitudinal axis X of article 10 and disposed on an underside of T-shaped substrate 77. Strap 80 could be made of the same materials making up leg portions 63, distal portion 65, and/or proximal portion 58, with the elastic strands 69 being oriented transversely to the longitudinal axis X, thereby rendering the strap 80 extensible in that transverse direction. Alternatively, strap 80 could be made of any elastomeric, extensible material available in the market, or even take the form of non-extensible strips or strings that may be tied to another one once a chosen level of tightness around the penis P is achieved. In a specific embodiment, strap 80 is made up of one or more layers of nonwoven material of a suitably chosen basis weight, laminated to an extensible, elastomeric substrate, such as the Conforma® 9800 material discussed above.

Strap 80 includes, in the illustrated example embodiment, a hook-type coupler 82 that cooperates with one or more loop-type couplers 84 disposed on an exterior surface of proximal portion 58 of sling 14. During application of article 10, the strap 80 is selectively extended by pulling on the part of the strap 80 that includes the hook-type coupler 82, wrapped around toward the diametrically opposite side of article 10, and made to engage one of the loop-type couplers 84. Alternative embodiments are contemplated in which the couplers 82, 84 are instead in the form of an adhesive tape and cooperating film surface or any other fastener that allows releasable coupling of one of the couplers to the other. Yet another embodiment is contemplated in which the hook-type coupler 82 engages directly to the external surface of the outer layer 28 of pouch 12 or directly to the external surface of proximal portion 58, in which case the cooperating coupler 84 takes the form of one of those external surfaces. Likewise, it is contemplated that coupler 82 may instead be a loop-type fastener cooperating with a hook-type coupler 84 on the external surface of outer layer 28 or proximal portion 58.

For embodiments using hook and loop fasteners for couplers 82, 84, suitable materials include those known in the trade under the names SoftGrip® or TexLoop®, both commercially available from Aplix of Le Cellier, France. For embodiments using a combination of adhesive tape and a cooperating film surface, a suitable material is a fastening tape-release tape combination known under the trade name Y-Bond®, commercially available from Avery Dennison of Mentor, Ohio. While not shown, it is contemplated that strap 80 may include a layer of skin-care composition similar to the layer 33 of skin-care composition described above with respect to pouch 12.

The material or material combination making up strap 80 are suitably chosen to provide the right balance between comfort to the wearer and the ability of the strap 80 to securely maintain article 10 in place, particularly the opening 21 of pouch 12 around penis P. In that regard, the inventors have found that, for most wearers, strap 80 is normally stretched in the longitudinal direction of strap 80 (i.e., generally along the length dimension of strap 80) by about 20% to about 30%, and more specifically by about 25%. The inventors have further found that the material or material combination making up strap 80 may be chosen such that the retraction force—in the longitudinal direction of strap 80—in the about 20 to 30% strain range does not exceed about 0.85 N. That level of retraction force in use strikes the right balance between comfort to the wearer (which makes low retraction forces desirable) and securely maintaining opening 21 of pouch 12 around penis P (which makes high retraction forces desirable) during normal wear.

The measurement discussed above is obtained following a standard force vs. strain (displacement) tensile test, which utilizes an Instron® machine. It should be noted, notwithstanding, that the relatively low level of retraction forces (such as those no greater than about 0.85 N) may be detrimental to the wearer's sense of security, leading him to believe that pouch 12 will not stay securely in place around penis P during wear—or that urine may escape through any gaps defined between penis P and opening 21 of pouch 12. Table 3, below, shows the average retraction force for three different materials or material combinations that were found suitable for strap 80.

TABLE 3
Average Retraction Force of Strap at
different strain levels (in Newtons)

| Strap | 20% | 25% | 30% |
|---|---|---|---|
| Construction | Strain | Strain | Strain |
| A | 0.21 | 0.32 | 0.43 |
| B | 0.25 | 0.31 | 0.37 |
| C | 0.6 | 0.72 | 0.82 |

In Table 3, above, the material or material combinations (“strap construction”) A, B, and C are the same as those described above with reference to Table 2.

While not shown, alternative embodiments are contemplated having no strap 80 at all but having instead an elasticized material or combination of materials at open end 20 of pouch 12, defining opening 21. In those embodiments, opening 21 would be manually expanded against the contraction forces of the elasticized material(s) to allow insertion of penis P. The elastic nature of the opening 21 would cause it to retract, thereby securing pouch 12, at its open end 20, against penis P.

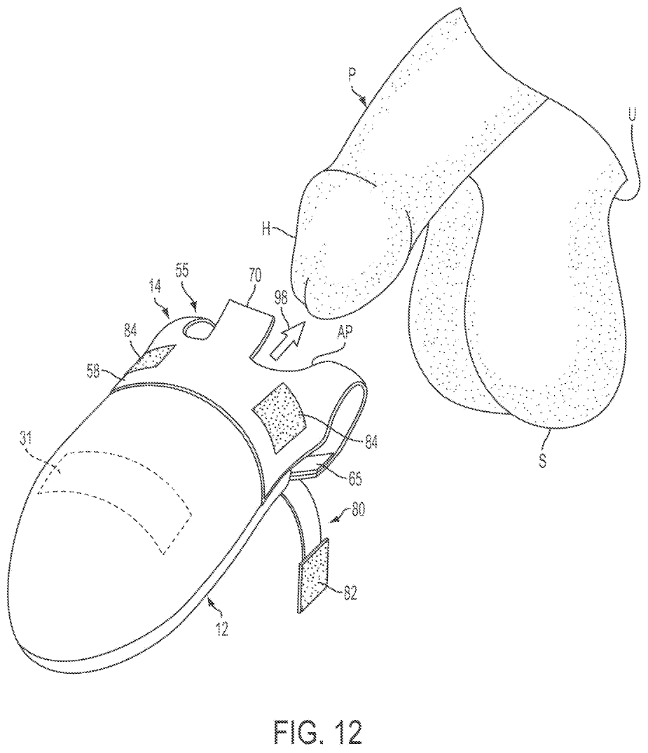

FIGS. 11 and 12 illustrate yet another feature that aids in the application (i.e., deployment) of the embodiment of that figure to the body of the wearer. Specifically, in that example embodiment, article 10 includes a pair of matching secondary couplers 91 and 93 that cooperate to temporarily secure loop portion 55 to the remainder of article 10 to signal to the wearer or his caregiver the direction of insertion of the penis P through the aperture AP defined by loop portion 55. Secondary couplers 91, 93 may take the form, for example, of cooperating hook and loop components of a hook-and-loop fastener. Securing loop portion 55 in place during application of article 10 also makes the application a one-handed operation for the wearer or his caregiver, further enhancing the overall experience.

In the illustrated embodiment, the secondary couplers 91 and 93 are respectively disposed on distal portion 65 of sling portion 55 and on a surface of second pull tab 75. When the article 10 is first presented to the wearer or his caregiver, the sling portion 55 is temporarily secured to the exterior-facing side of second pull tab 75, and the position and orientation of sling portion 55 are thereby defined by that temporary coupling, leaving only one side of the aperture AP defined by sling portion 55 available for insertion of penis P. Arrow 96 in FIG. 11 illustrates temporary engagement of second couplers 91 and 93 to one another to thereby yield the form in which article 10 may be presented to the wearer for the first time.

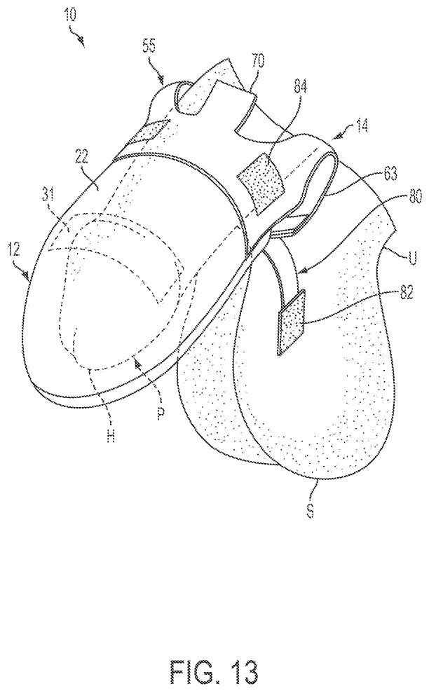

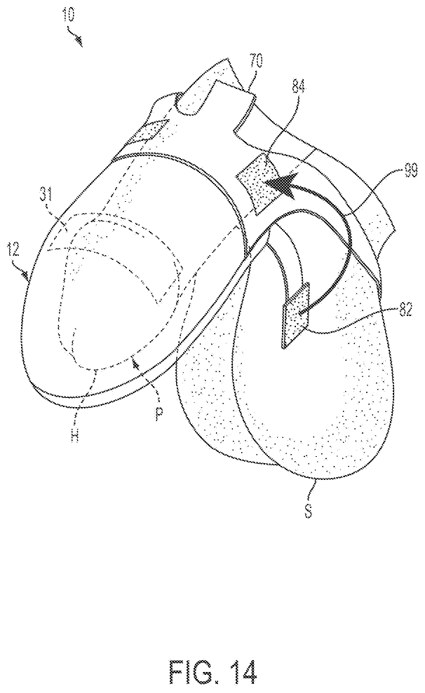

FIGS. 7, 12, 13, 14, and 15 illustrate an example application of article 10 onto the pertinent part of the body of the wearer. Lateral forces (arrows 32 in FIG. 7) are exerted onto the sides of pouch 12, which causes the opening 21 at the first end 20 to expand. The penis P is then inserted (arrow 98 in FIG. 12) through the aperture AP defined by loop portion 55, with the article 10 oriented such that distal portion 65 is more or less on the same plane as the underside of penis P. Article 10 is then moved toward the base of penis P by pulling on the one or more pull tabs 70, 75 and the secondary couplers 91, 93 disengaged from one another to free sling portion 55 for movement. FIG. 13 shows the penis P having been fully inserted into the interior 22 of pouch 12 through opening 21 (FIG. 7). Next, the sling portion 55 is held and extended downwardly and under scrotum S, until distal portion 65 is securely anchored against the underside U of scrotum S, as shown in FIG. 14.

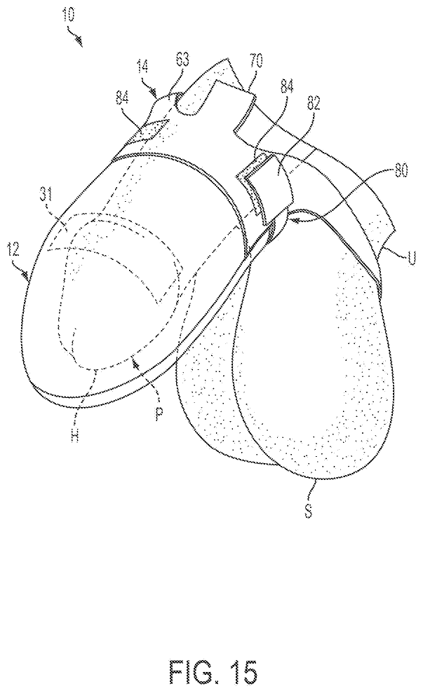

The wearer or caregiver then proceeds to extend strap 80 and bring same around to the diametrically opposite side of article 10 (arrow 99), and to secure hook-type coupler 82 to one of the loop-type couplers 84 (FIGS. 14 and 15). In that regard, the wearer or caregiver selects the loop-type coupler 84 that provides the highest level of comfortable securement around the penis P i.e., the desired balance of snug fit and comfort for the wearer. Alternative embodiments are contemplated in which the strap 80 is guided through an eyelet or similar, to make it easily reachable to the wearer and/or caregiver during application of article 10. Depending on the wearer's penis shape and/or dimensions, a very tight fit may not necessarily feel comfortable to the wearer. It is contemplated, in that regard, that article 10 may instead have more than two loop-type couplers 84 or may instead have, for example, a continuous strip of cooperating loop-type material (or a continuous strip of hook-type material if coupler 82 is of the loop-type) along the width of article 10. Alternatively, hook-type coupler 82 may instead be configured to mechanically engage and thereby fasten directly to any external surface of pouch 12 or sling 14.

Alternative embodiments are contemplated in which the secondary couplers 91, 93 are replaced by a non-releasable connector such as a piece of readily tearable thread, for example and without limitation, that secures loop portion 55 in place until easily torn by the wearer during application of the article 10 as described above. Yet other embodiments that are contemplated include adhesive to provide temporary securement of loop portion 55 in place until the moment of application, or a hook-type component of a hook-and-loop fastener on distal portion 65, as shown in the figures, configured to be releasably attached directly to the surface of second pull tab 75, directly to the surface of T-shaped substrate 77, or to a surface of pouch 12. Yet other embodiments may include no secondary couplers 91, 93 at all, and still be considered to fall within the scope of the present disclosure.

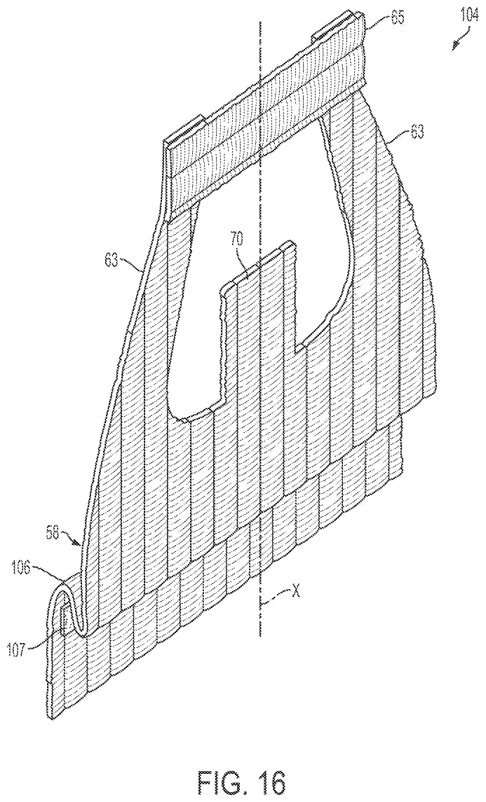

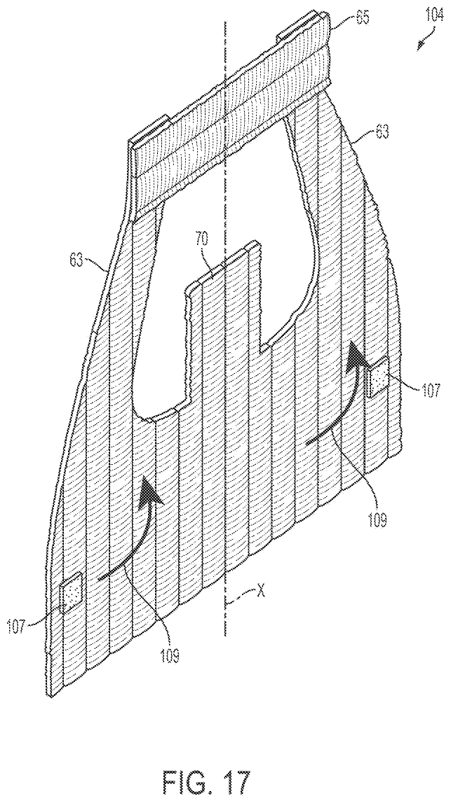

With continued reference to the preceding figures and further referring to FIGS. 16 and 17, an alternative embodiment is shown of a sling 104 that may be selectively elongated to provide for an overall greater length of the article of which it forms part. For ease of understanding, like reference numerals in those figures and in the description thereof refer to similar features in the embodiments of the preceding figures, the structures, relationships, functionalities, and/or contemplated variations of which may be referred to for an understanding of this embodiment in FIGS. 16 and 17 as well. Sling 104 includes a fold 106 in the proximal portion 58 that is secured in place by a pair of fasteners 107 that may be of the releasable type, such as a hook-and-loop type fasteners, or non-releasable type fasteners such as adhesive patches or tearable thread that, once disengaged (e.g., torn), may not be reengaged to secure the fold 106 in place. It is contemplated that certain embodiments of the type described herein may have fasteners 107 in a number other than two, and still fall within the scope of the present disclosure.

If desired, the wearer of his caregiver may select to disengage/tear fasteners 107 to undo fold 106, thereby increasing the length (the dimension along axis X) of sling 104, as suggested by arrows 109 in FIG. 17. The optional elongation of sling 104 provided by undoing of fold 106 results in greater comfort to the wearer, particularly when the size and/or natural movement of his penis P requires a longer article. Advantageously, the selective elongation of sling 104 and of the article as a whole, provides the manufacturer of that article the ability to sell a single type/size of article for all wearers, rather than needing to offer various sizes for sale. This provides for efficiency in the marketplace. However, it should be noted that the presence of fold 106 may be detrimental to the commercial appeal of the article. Specifically, fold 106 may be perceived as a defect in what would be otherwise expected to be a flat sling 104. Further, the presence of fold 106 increases the overall thickness of sling 104, at least in the area that includes that fold 106, which may increase the overall bulkiness of the article during wear and/or cause discomfort to the wearer.

An alternative embodiment (not shown) is contemplated in which selective elongation of pouch 12 is provided by having at least a portion of pouch 12 (e.g., the area adjacent open end 20) or the proximal portion 58 of sling 14 made up of an easily stretchable and permanently deformable material that may be stretched out (generally in the longitudinal dimension of article 10) to a desired length. In that type of embodiment, a minimum predetermined force would be required for such selective elongation, to prevent inadvertent stretching during wear. A suitable stretchable and permanently deformable material of the type described above is a film laminate known in the trade as Ela-mask™, commercially available from Texol Srl of Alanno, Italy.

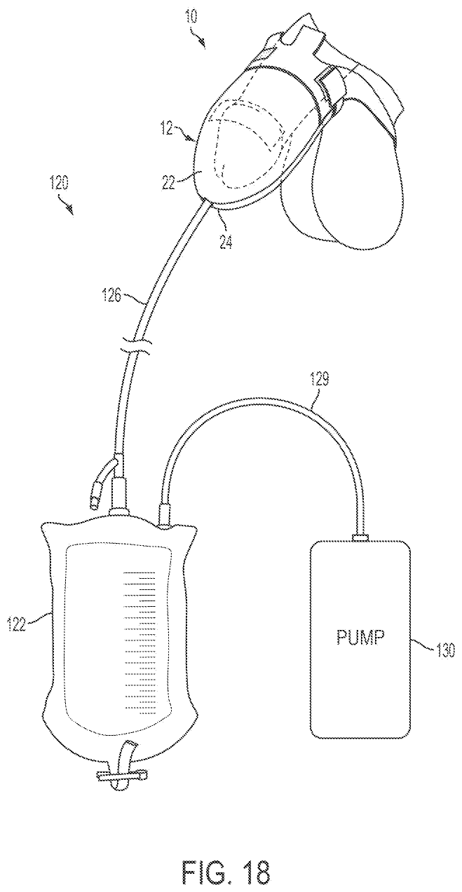

FIG. 18 illustrates an embodiment that permits the article in the preceding figures to become part of a catheter 120. In FIG. 18, like reference numerals refer to similar features in the embodiments of the preceding figures and contemplated variations thereof. Catheter 120 includes an incontinent article such as article 10 in FIGS. 1-7, fluidly connected to a storage bag 122 through a hose 126 that is fluidly coupled to the closed end 24 of pouch 12 of article 10. Storage bag 122 is in turn fluidly connected, through a second hose 129, to an electrical pump 130 that, when energized (e.g., turned on), creates a vacuum space (i.e., negative pressure) in the interior of bag 122, which is effective to pull urine (or other fluids) from interior 22 of pouch 12 into storage bag 122. The urine storage capacity provided by article 10 in catheter 120 complements the overall urine storage capacity provided by storage bag 122.

In a contemplated variation, catheter 120 includes an incontinent article similar in most respects to article 10, as shown and described above, but which does not include an absorbent core 30 (FIG. 5) or any other component configured to store urine. In that embodiment, the urine storage capacity is solely provided by the storage bag 122. Yet other contemplated embodiments include an incontinent article similar in most respects to article 10 (as shown and described above), but which has only a relatively small storage capacity. Embodiments of that type may, for example, include a storage core that is configured with a storage capacity that is less than about 85 ml, specifically less than about 50 ml in some embodiments, and more specifically greater than 0 ml but less than about 30 ml in specific embodiments.

In any of those embodiments, further, the outer layer of the pouch of the article may or may not be hydrophobic, since urine is not likely to accumulate in the interior 22. Also, in those contemplated embodiments, the inner layer of the pouch may be hydrophobic. In those embodiments at least a portion of the pouch, such as the area adjacent the open end, may have an elastic material such that contraction of that elasticized material is effective to secure the open end of the pouch about penis P, thereby minimizing or altogether preventing leakage of urine through opening 21 (FIG. 2).

It is contemplated that the various embodiments and features described above, including variations thereof, may be combined with one another, even if a specific combination of features is not shown in one of the accompanying figures or described in association with one particular embodiment. From the above disclosure of the general principles of the present invention and the preceding detailed description of exemplifying embodiments, those skilled in the art will readily comprehend the various modifications to which this invention is susceptible. Accordingly, this invention is intended to be limited only by the scope of the following claims and equivalents thereof.

What is claimed is:

1. An incontinence article for a male having a penis and a scrotum and extending along a longitudinal axis, the article comprising:
a pouch including one or more layers of nonwoven material and configured to envelop the penis, said pouch having a first open end and a second closed end longitudinally opposed said first open end; and
a sling connected to said pouch adjacent said first open end of said pouch, said sling including a loop portion configured to fit around an underside of the scrotum and the penis, said loop portion being extensible in at least a generally circumferential direction thereof.

2. The incontinence article of claim 1, wherein said pouch includes an absorbent core having superabsorbent material.

3. The incontinence article of claim 2, wherein said absorbent core includes fluff pulp in an amount not exceeding about 20% by weight of said absorbent core.

4. The incontinence article of claim 1, wherein said loop portion includes at least one elastic strand extending in the generally circumferential direction of said loop portion.

5. The incontinence article of claim 1, wherein said sling includes a proximal portion connected to said pouch, a pair of leg portions extending from said proximal portion, and a distal portion spanning between respective terminal ends of said leg portions in a direction transverse to the longitudinal axis of the article, said proximal, leg, and distal portions jointly defining said loop portion of said sling.

6. The incontinence article of claim 5, wherein said proximal portion is integrally formed with said pouch.

7. The incontinence article of claim 5, wherein said leg portions and said distal portion have respective length dimensions, each of said leg and distal portions being extensible along said respective length dimension thereof.

8. The incontinence article of claim 7, wherein each of said leg portions and said distal portion includes a plurality of elastic strands extending generally along said respective length dimension thereof.

9. The incontinence article of claim 5, wherein said leg portions and said distal portion are integrally formed with one another.

10. The incontinence article of claim 9, wherein said leg portions and said distal portion are formed from a multidirectional elastomeric material.

11. The incontinence article of claim 5, wherein said proximal portion is extensible in a direction generally parallel to the longitudinal axis of the article.

12. The incontinence article of claim 5, wherein said proximal portion includes a releasable fold extending in a direction transverse to the longitudinal axis of the article, said fold secured in place by a fastener configured to be selectively disengaged to permit extension of the overall dimension of the article along the longitudinal axis thereof.

13. The incontinence article of claim 1, further comprising a pull tab adjacent said first open end of said pouch and configured for pulling said pouch toward the base of the penis.

14. The incontinence article of claim 1, further comprising a strap adjacent said first open end and configured for securing said pouch around the penis, said strap including a hook-and-loop element adapted to mechanically fasten to an external surface of said pouch or said sling.

15. The incontinence article of claim 1, wherein said first open end of said pouch is the only opening configured for insertion and removal of the penis into/from said pouch.

16. The incontinence article of claim 1, wherein said one or more layers of nonwoven material include a hydrophilic layer and a hydrophobic layer, said hydrophilic and hydrophobic layers in an overlaying relationship with one another.

17. The incontinence article of claim 1, wherein said pouch includes a stiffening element between said first and second ends of said pouch, said stiffening element configured to minimize folding of said one or more layers of nonwoven material during wear of the article.

18. The incontinence article of claim 1, wherein said loop portion of said sling includes a layer of lotion configured to lower a level of friction between said loop portion and the scrotum during wear.

19. An incontinence article for a male having a penis and a scrotum and extending along a longitudinal axis, the article comprising:
a pouch including one or more layers of nonwoven material and configured to envelop the penis, said pouch having a first open end and a second closed end longitudinally opposed said first open end; and
a sling connected to said pouch adjacent said first open end of said pouch, said sling including a loop portion configured to fit around an underside of the scrotum and the penis, said loop portion being extensible in at least a generally circumferential direction thereof, wherein:
said pouch includes a pocket selectively openable to permit removal of the penis therethrough, said pocket having an open condition in which access to an interior of the pouch is permitted and a second condition in which said access is not permitted.

20. An incontinence article for a male having a penis and a scrotum and extending along a longitudinal axis, the article comprising:
a pouch configured to envelop the penis and including a layer of hydrophilic nonwoven material, a layer of hydrophobic nonwoven material overlaying said layer of hydrophilic nonwoven material, and a stiffening element disposed between said layers, said pouch having a first open end and a second closed end longitudinally opposed said first open end, said stiffening element being located between said first and second ends of said pouch and configured to minimize folding of said layers of nonwoven material during opening of said first open end;
a sling connected to said pouch adjacent said first open end of said pouch, said sling including a loop portion configured to fit around an underside of the scrotum and the penis, said loop portion including a plurality of elastic strands oriented so as to make said loop portion extensible in a generally circumferential direction thereof;
a pull tab adjacent said first open end of said pouch and configured to permit pulling of said pouch toward a base of the penis; and
a strap adjacent said first open end and configured for securing said pouch around the penis, said strap including a hook and/or loop element adapted to mechanically fasten to an external surface of said pouch or said sling.
